(19) United States
(12) Patent Application Publication
(10) Pub. No.: US 2008/0275492 A1
(43) Pub. Date: Nov. 6, 2008

(54) EXTENDED DURATION MEDICAL FILTER WITH BIO-ABSORBABLE BARBS

(76) Inventor: Victor Farmiga, Putnam Valley, NY (US)

Correspondence Address: RONALD L. HOFER, 122 LINDBERGH LANE, MOORESVILLE, NC, 28117, US

(21) Appl. No.: 11/742,729

(22) Filed: May 1, 2007

(51) Int. Cl. A61F 2/01

(52) U.S. Cl. 606/200

(57) ABSTRACT

A removable, radially compressible and expandable medical filter for placement in a tubular body passage such as a blood vessel. In radially expanded form, the medical filter has a main structure with a plurality of barbs extending radially outwardly from the main structure. The barbs are spaced circumferentially around the structure and are adapted to stand-off the main structure from the wall of a tubular body passage such as a blood vessel after the filter has been implanted and allowed to expand therein. The barbs also are adapted to anchor the main structure in the tubular passage to prevent migration of the filter in the passage. The barbs are comprised of a bio-absorbable material.

### BACKGROUND AND SUMMARY OF THE INVENTION

The present invention relates to a medical filter which can be placed inside a blood vessel or other body passage. More specifically, the present invention relates to a medical filter which can be placed inside a blood vessel or other body passage and which has barbs made of a bio-absorbable material.

Medical filters, such as vena cava filters, are emplaced inside blood vessels or other body passages for the purpose of intercepting thrombus or particles while allowing free flow of blood in the vessels or other body fluid in the body passages. Medical filters often are emplaced and never retrieved, remaining effective during their time in place and remaining permanently in place. However, while a medical filter may be implanted permanently, it may be desirable to retrieve the filter. If retrieval is desired, it should be retrieved as easily as possible with minimal trauma to the vessel or body passage. If the filter has just been implanted, retrieval may be relatively easy. But in some cases it may be desirable to remove or retrieve a medical filter after it has been in place for an extended period of time, for example, after it has been in place for more than two weeks.

Generally speaking, it is known to emplace or deliver medical filters in body passages as well as to remove the medical filters following their implantation. For example, it is known to implant and retrieve vena cava filters in blood vessels although retrieval of vena cava filters after they have been in place for an extended period of time can be challenging. Vena cava filters commonly have a network of interconnected elements and after implantation, self-expand radially in the blood vessel to a size matching the anatomy of the associated blood vessel. Vena cava filters commonly anchor themselves in the blood vessel to resist migration therein and have filter structure which is in contacting relationship with the wall of the blood vessel. However, immediately after being implanted in the blood vessel, there is a proliferation of tissue cell growth of the vessel which leads to endothelialization or growth of cellular structure of the vessel around part of the filter structure. This growth can makes it challenging to remove the filter without trauma to the wall of the vessel.

In light of the challenge posed by removal of an extended duration medical filter, it would be beneficial to have an improved medical filter which facilitated removal thereof with minimal trauma even after extended duration, if such later removal is desired. It would also be desirable to have a practical and economical medical filter. Furthermore, it would be desirable to have a medical filter which can be implanted and removed from either a femoral or jugular approach and which can be adapted for use with a wide variety of specific filter basket designs.

Accordingly, the present invention provides an improved, removable, radially compressible and expandable medical filter for placement in a tubular body passage such as a blood vessel. In radially expanded form, the medical filter of the present invention has a main structure with a plurality of barbs extending radially outwardly from the main structure. The barbs are spaced circumferentially around the structure and are adapted to stand-off the main structure from the wall of a tubular body passage such as a blood vessel after the filter has been implanted and allowed to expand therein. The barbs also are adapted to anchor the main structure in the tubular passage to prevent migration of the filter in the passage. The barbs are comprised of a bio-absorbable material.

Thus, in accordance with the present invention, after a filter of this invention has been implanted in a vessel or the like, it is contemplated that the bio-absorbable barbs will initially secure the filter structure to the vessel and will stand-off the main structure of the filter from the vessel wall during the initial proliferation of tissue re-growth occurring after its implantation in the vessel. Then the barbs will be bio-absorbed, leaving the main structure secured in the vessel but with an amount of tissue over the structure allowing its removal from the vessel.

In accordance with the method of the present invention, a medical filter of the present invention is implanted in a body passage, the barbs are bio-absorbed therein and then the filter is removed therefrom by using a retrieval catheter.

Further understanding of the present invention will be had from the following description taken in conjunction with the accompanying drawings and appended claims.

### BRIEF DESCRIPTION OF THE DRAWINGS

FIG. 1 is a perspective view, showing a preferred embodiment of a medical filter of the present invention;

FIG. 2 is a side elevation of the preferred embodiment of FIG. 1 shown in radially expanded form in a blood vessel immediately after implantation therein, the blood vessel being shown in partial section;

FIG. 3 is a side elevation of the preferred embodiment of FIG. 2 in the blood vessel after an initial proliferation of tissue growth has occurred therein, the blood vessel being shown in partial section;

FIG. 4 is side elevation of the preferred embodiment of FIG. 3 in the blood vessel after the barbs of the filter have been bio-absorbed and the initial proliferation of tissue growth has ended, the blood vessel being shown in partial section; and

FIG. 5 is a side elevation, with portions broken away, illustrating removal of the medical filter of FIG. 4 from the blood vessel, the blood vessel being shown in partial section;

### DETAILED DESCRIPTION OF THE INVENTION

The following description of the preferred embodiments of the present invention is intended to be merely illustrative in nature, and as such, is not intended to limit in any way the present invention, its application, or uses. Numerous modifications may be made by those skilled in the art without departing from the true spirit and scope of the invention. For example, the present invention may be used in veins but is also useful in other body passages. Also, while the preferred embodiment of the present invention is shown with bio-absorbable barbs on a specific, preferred, main filter structure, it will be appreciated by those skilled in the art that other suitable designs of main filter structures come within the spirit and broad scope of this invention.

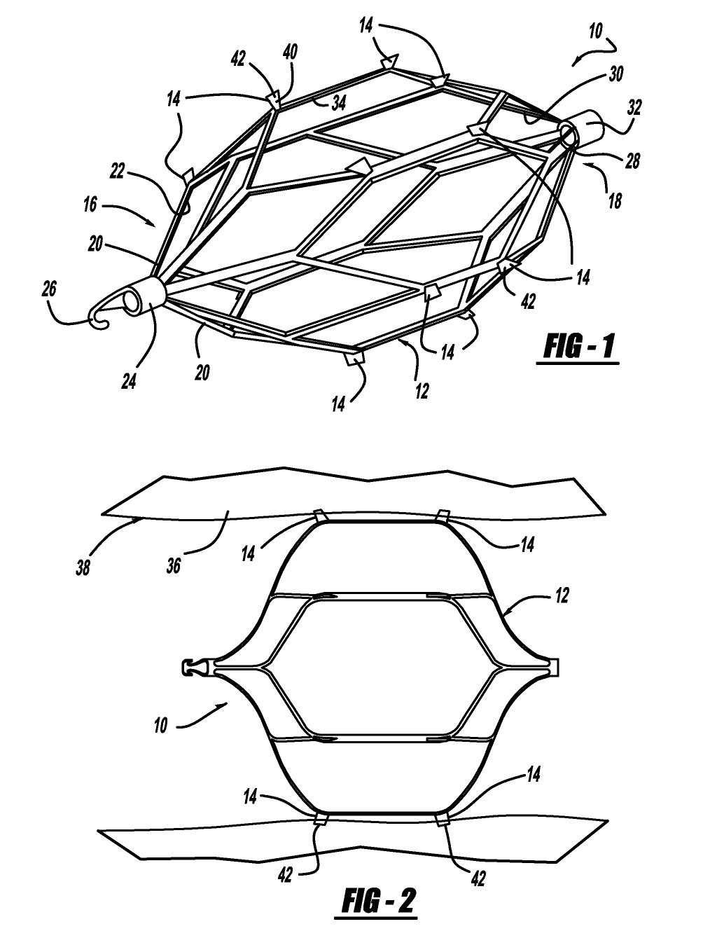

Now referring to FIG. 1, a preferred embodiment of a medical filter of the present invention is shown and indicated generally by the numeral 10. FIG. 1 shows medical filter 10 in radially expanded form. Broadly speaking, medical filter 10 has a main structure 12 which carries a plurality of bio-absorbable ski barbs 14 which extend radially outwardly from main structure 12.

Main structure 12 has first and second filter baskets 16 and 18. Filter basket 16 has a plurality of filter elements 20 which form diamond shaped openings 22. The radially inward end of each element 20 is connected to first central member 24 which carries retrieval hook 26. Filter basket 18 has a plurality of filter elements 28 which form diamond shaped openings 30. The radially inward end of each filter element 28 is connected to second central member 32. Although not shown in the figure, it will be appreciated that second central member 32 may carry a retrieval element identical or different than retrieval hook 26. A plurality of struts 34 extend between filter basket 16 and filter basket 18, each strut 34 being connected to a radially outward end of a filter element 20 and a radially outward end of a filter element 28.

A plurality of bio-absorbable ski barbs 14 extend radially outwardly from struts 34. It will be appreciated by those skilled in the art that the structure of main structure 12 is generally that disclosed in U.S. Pat. No. 6,989,021 B2, Jan. 24, 2006 to Bosma et al. for “Retrievable Medical Filter,” the disclosure of which is specifically incorporated by reference herein. Of course, the anchoring ski barbs shown in the Bosna et al. patent (as anchoring barbs 26 therein) are not required in the present invention which has bio-absorbable ski barbs 14. Thus, in the present invention, struts 34 of main structure 12 extend generally parallel to each other and to the longitudinal axis of main structure 12 of filter 10. Struts 34 are also spaced circumferentially about main structure 12 in the outline of a generally tubular shape. Bio-absorbable ski barbs 14 are located on struts 34 and extend radially outwardly therefrom. Preferably each strut 34 carries a pair of ski barbs 14 with one of each pair located at or near the transition between strut 34 and associated filter elements 20 and 28. As best shown in FIG. 2, bio-absorbable ski barbs 14 stand off main structure 12 from wall 36 of vessel 38 and anchor filter 10 therein as described in more detail below.

Bio-absorbable ski barbs 14 are shown in their preferred form as being elongated in the axial direction to provide a length, or runner 40, with a surface 42 adapted to come into contacting relationship with wall 36 of an associated vessel 38. Ski barb runner surface 42 minimizes any tendency of ski barb 14 to pierce wall 36 of vessel 38. Of course, ski barbs 14 may be modified to have other geometric shapes so long as they function in accordance with the present invention.

Ski barbs 14 are comprised of a bio-degradable material, which of course must also be biocompatible. Suitable bio-degradable materials are well-known and include carbon steel or polymeric materials. Suitable polymeric materials include alpha polyesters such as polylactide (PLA) and polyglycolide (PGA), polydioxanone, polycaprolactone, polyhydroxybutyrate and poly(aminoacids). Copolymers and terpolymers may also be used, such as a terpolymer of poly-(L-lactide/D-lactide/glycotide). Of course, the ski barb material must be mechanically secured or bonded to main structure 12, preferably on struts 34 thereof as shown in the figures. Suitable methods for such securement will be apparent to those skilled in the art and are dependent upon the particular bio-degradable material used.

It will be further appreciated by those skilled in the art that main structure 12 is intended to be radially compressible and is normally delivered to its useful site in compressed form in a delivery catheter or the like and then allowed to expand radially into operative form. Filter baskets 16 and 18 of main structure 12 are comprised of several separate components: filter elements 20, first central members 24, retrieval hook 26, filter elements 28, and central member 32. All of these components can be separate pieces fastened together or can be integrally formed as by cutting from a single tube. Thus, the main structure 12 must be made of a resilient material which can be compressed to a smaller diameter and cylindrical form for insertion into the lumen of a delivery catheter and then expanded radially to the form illustrated in the figures for use in a vessel or body passage.

Main structure 12 can be made of any suitable material using a variety of methods. Suitable conventional materials and methods are well known in the art. Nitinol and stainless steel are examples of suitable materials but other materials may be used so long as the material has the desired characteristics of strength, resilience, flexibility, biocompatibility and endurance and is suitable for the particular manufacturing technique employed. It is, of course, required that the material employed be capable of expanding to the desired shape upon ejection from the delivery catheter and reduction in diameter when withdrawn into the lumen of the retrieval catheter. Thus, the material must also be sufficiently resilient to accomplish the compression and expansion required for use of filter 10.

Suitable methods of manufacture of filter 10 include cutting a pattern into a tube to enable expansion of the tube into the desired main structure and optionally arms as well. Another suitable method is forming the main structure and arms from separate strips or wires and then joining the respective parts together by suitable methods which are well known in the art.

Of course, it will be appreciated by those skilled in the art that the main structure can have many different designs and may be modified within the spirit and scope of the present invention. For example, the main structure can have additional struts and can be symmetrical or asymmetrical in form.

Having described a medical filter of the present invention, further understanding of the unique character and advantages of the present invention will be had by the following description of its intended operation. Thus, it is intended that filter 10 will be deployed or implanted in a body passage such as blood vessel 38 by positioning filter 10 in the vessel using a delivery catheter with a sheath and then withdrawing the sheath, whereupon main structure 12 of filter 10 expands radially to the form illustrated in FIG. 2. Upon expansion of main structure 12, surfaces 42 of runners 40 are biased against wall 36 of blood vessel 38 and filter 10 can be left in the vessel as a permanent treatment. Bio-absorbable ski barbs 14 prevent migration of filter 10 in vessel 38.

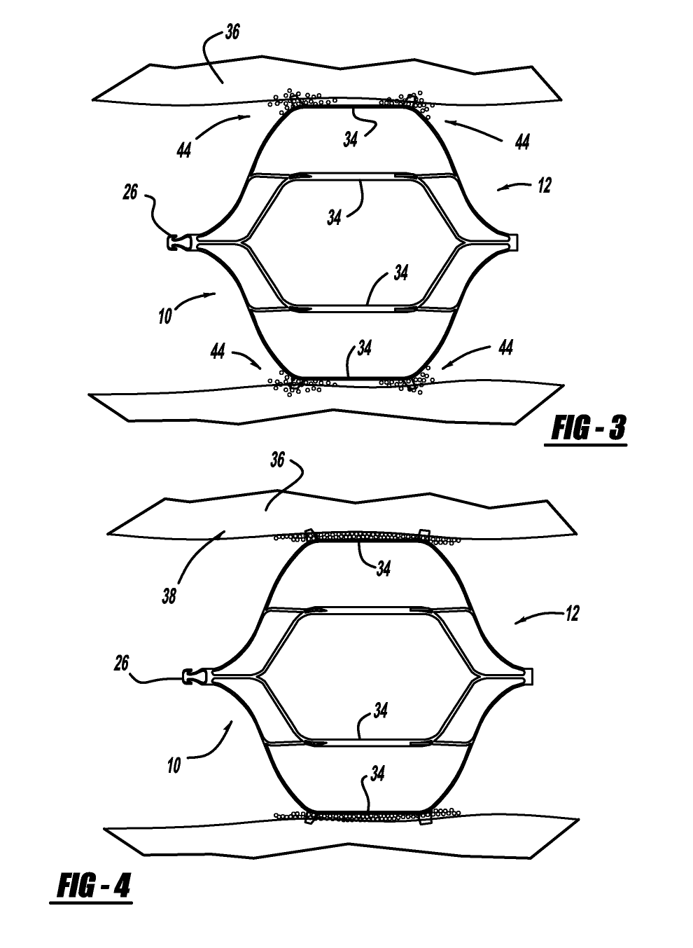

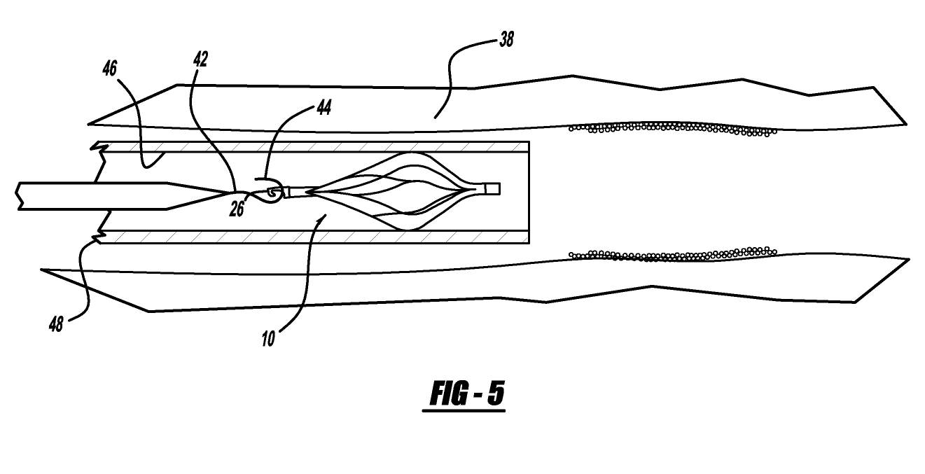

Bio-absorbable ski barbs 14 initially act to secure filter 10 to vessel wall 36, provide appropriate orientation of filter 10, and stand off main structure 12 of filter 10 from vessel wall 36 to keep it away from post-treatment proliferative tissue regrowth. FIG. 2 illustrates the initial relationship of filter 10 and vessel 38 showing no growth of tissue over ski barbs 14. Then tissue 44 begins to grow over bio-absorbable ski barbs 14 as illustrated in FIG. 3. And after a period of time, the proliferative tissue regrowth process subsides and bio-absorbable ski barbs 14 are absorbed and disappear as is illustrated in FIG. 4 with main structure 12 of filter 10 biased radially outwardly so that struts 34 are biased against wall 36 of vessel 38. Since struts 34 are not encapsulated in tissue 40, as best shown in FIG. 4, in the event it is desired to remove filter 10 from vessel 38, retrieval hook 26 can be snared and pulled into a sheath or guiding catheter device. Once emplaced in a body passage such as a vein, in many cases there will be no desire to retrieve filter 10 from the vessel. However, in some cases there will be a desire to retrieve filter 10 from the vessel and in such cases the present invention provides a filter 10 which facilitates retrieval since there is minimal tissue growth over struts 34.

FIG. 5 illustrates the step of retrieving filter 10. Filter 10 is shown in place in vessel 38. Retrieval wire 42 with snare 44 is shown having been advanced through vessel 38 to a location proximate to filter 10. Snare 44 is shown having captured retrieval hook 26. As will be appreciated by the skilled artisan, snare 44 is withdrawn into lumen 46 of retrieval catheter 48 to radially collapse filter 10 therein for retrieval from vessel 38.

While preferred embodiments of the present invention have been specifically described above, it will be appreciated by those skilled in the art that the present invention is subject to variations and modifications. For example, main structure 12 can be modified to have different designs within the scope of this invention and ski barbs 14 can be modified to have different geometric shapes within the scope of this invention which is intended to be limited only by the following claims.

What is claimed is:

1. A removable, radially compressible and expandable medical filter for placement in a tubular body passage such as a blood vessel, the medical filter having in radially expanded form, a main structure and a plurality of bio-absorbable barbs spaced circumferentially around said main structure and extending radially outwardly therefrom

2. A filter as set forth in claim 1, wherein said barbs are adapted to stand-off said main structure from a wall of a tubular body passage.

3. A filter as set forth in claim 1, wherein said barbs are adapted to anchor said main structure in a tubular passage to prevent migration of the filter in the passage.

4. A filter as set forth in claim 1, wherein said main structure has first and second filter baskets connected to longitudinally extending struts extending therebetween.

5. A filter as set forth in claim 1, wherein said barbs extend from said struts.

6. A filter as set forth in claim 1, wherein said barbs extend from said struts where said struts are connected to said filter baskets.

7. A filter as set forth in claim 1, wherein said barbs are comprised of a bio-absorbable polymer.

8. A filter as set forth in claim 7, wherein said polymer is selected from the group consisting of polylactide, polyglycolide, polydioxanone, polycaprolactone, polyhydroxybutyrate, and poly(aminoacid) polymers, copolymers and terpolymers thereof.

9. A filter as set forth in claim 6 wherein said barbs are mechanically connected to said struts.

10. A filter as set forth in claim 6, wherein said barbs are bonded by adhesive to said struts.

11. A filter as set forth in claim 6, wherein said barbs are adhered to said struts.

12. A filter as set forth in claim 1, wherein said main structure is comprised of a material selected from nitinol and stainless steel.

13. In combination, a blood vessel having a tubular wall and a medical filter, said medical filter being expanded in said blood vessel and having a main structure and a plurality of bio-absorbable barbs spaced circumferentially around said main structure and extending radially outwardly therefrom to thereby stand off said main structure from said wall of said blood vessel.

14. A filter as set forth in claim 13, wherein said barbs are adapted to anchor said main structure in said blood vessel to prevent migration of said filter therein.

15. A filter as set forth in claim 13, wherein said main structure has first and second filter baskets connected to longitudinally extending struts extending therebetween and said barbs extend from said struts.

16. A filter as set forth in claim 15, wherein said barbs extend from said struts at a point where said struts are connected to said filter baskets.

17. A filter as set forth in claim 13, wherein said barbs are comprised of a bio-absorbable polymer.

18. A filter as set forth in claim 17, wherein said polymer is selected from the group consisting of polylactide, polyglycolide, polydioxanone, polycaprolactone, polyhydroxybutyrate, and poly(aminoacid) polymers, copolymers and terpolymers thereof.

19. A method of treatment of a patient having a tubular blood vessel with a wall, said method comprising the steps of implanting in said tubular blood vessel an elongated medical filter which is radially compressible and radially expandable, said filter having in radially expanded form, a main structure and a plurality of bio-absorbable barbs spaced circumferentially around said main structure and extending radially outwardly therefrom, said filter expanding to expanded form after being implanted in said blood vessel whereupon said barbs stand off said main structure from the wall of said blood vessel.
